(19) United States
(12) Patent Application Publication
(10) Pub. No.: US 2018/0087116 A1
(43) Pub. Date: Mar. 29, 2018

(54) METHODS AND BIOSENSORS FOR TUMOR DETECTION

(71) Applicants: Mahmoud Amouzadeh Tabrizi, Tehran (IR); Mojtaba Shamsipur, Tehran (IR); Reza Saber, Tehran (IR); Saeed Sarkar, Tehran (IR)

(72) Inventors: Mahmoud Amouzadeh Tabrizi, Tehran (IR); Mojtaba Shamsipur, Tehran (IR); Reza Saber, Tehran (IR); Saeed Sarkar, Tehran (IR)

(21) Appl. No.: 15/818,767

(22) Filed: Nov. 21, 2017

Related U.S. Application Data
(60) Provisional application No. 62/457,827, filed on Feb. 11, 2017.

(51) Int. Cl. C12Q 1/6886

(52) U.S. Cl. CPC C12Q 1/6886

(57) ABSTRACT

A method for tumor marker detection is disclosed. The method includes preparing a biosensor, forming a reference solution by adding the biosensor to a buffer solution, measuring a first fluorescence intensity of the reference solution, forming a mixture by adding a suspicious biological solution to the reference solution, measuring a second fluorescence intensity of the mixture, and detecting a presence of a tumor marker responsive to a difference between the first fluorescence intensity and the second fluorescence intensity. The biosensor preparation includes forming a functionalized nanomotor by functionalizing a nanomotor with an aptamer, forming a blocked functionalized nanomotor by blocking gaps between functionalized parts of the functionalized nanomotor with a blocking agent, and attaching a fluorescence probe to the blocked functionalized nanomotor.

### CROSS-REFERENCE TO RELATED APPLICATION

This application claims the benefit of priority from pending U.S. Provisional Patent Application Ser. No. 62/457,827, filed on Feb. 11, 2017, and entitled “FABRICATION OF SMALL AND COST-EFFECTIVE NANOROD-SHAPED MOTOR AND APPLICATIONS THEREOF FOR SENSING VEGF165 TUMOR MARKER,” which is incorporated herein by reference in its entirety.

### SPONSORSHIP STATEMENT

This application has been sponsored by Iran Patent Office, which does not have any rights in this application.

### TECHNICAL FIELD

The present disclosure generally relates to a biosensor and a method for detecting tumor biomarkers, and particularly, to a biosensor based on nanomotors and a method for detecting tumor biomarkers using the biosensor.

### BACKGROUND

Determination of tumor markers such as VEGF165, MUC-1, MCF-7 and HER-2 with aptamer-based methods is one of the interesting pathways for the effective, selective and fast determination of cancer diseases. Up to now, a variety of optical, surface plasmon resonance, piezoelectric micro cantilever, quartz crystal microbalance, field-effect transistor, and ELISA based sensors have been reported for the assay of cancer biomarkers. However, these methods are mainly time-consuming, complex, and expensive operations.

To overcome these shortcomings, the fabrication of aptamer based sensing devices (aptasensors) for determination of tumor markers (such as VEGF165, mucin 1 (MUC1), Michigan Cancer Foundation-7 (MCF-7) and human epidermal growth factor receptor 2 (HER-2)) are of particular interest, in part due to their high selectivity, sensitivity and feasibility of quantification. Due to their sensitivity and specificity, aptasensors are suitable for the detection of low levels of tumor markers. Several platforms or substrates have been used for immobilization of the aptamer in aptasensors, for example, BSA-gold nanoclusters/ionic liquid nanocomposites, microtube engines, graphene-poly(amidoamine)/gold nanocomposite, etc. However, measurement procedures of such aptasensors involve additional cumbersome techniques, devices and apparatus which are complicated in some cases, for example, electrochemical techniques that are generally time-consuming and require operators with a high level of experience.

Hence, there is a need for simple, cost-effective and time-saving methods and biosensors to achieve a fast and exact way for detecting tumor markers. Also, there is a need for a biosensor and a method using thereof without any needs for further analysis or additional devices and apparatus for detecting tumor markers.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary method for tumor marker detection. The method may include preparing a biosensor, forming a reference solution by adding the biosensor to a buffer solution, measuring a first fluorescence intensity of the reference solution, forming a mixture by adding a suspicious biological solution to the reference solution, measuring a second fluorescence intensity of the mixture, and detecting a presence of a tumor marker responsive to a difference between the first fluorescence intensity and the second fluorescence intensity. Preparing the biosensor may include forming a functionalized nanomotor by functionalizing a nanomotor with an aptamer, forming a blocked functionalized nanomotor by blocking gaps between functionalized parts of the functionalized nanomotor with a blocking agent, and attaching a fluorescence probe to the blocked functionalized nanomotor.

In an exemplary implementation, the nanomotor may include a nanorod with a diameter of less than about 50 nm and a length of less than about 100 nm. The nanorod may include a first segment that may include Gold (Au), a second segment that may include a metal, and a third segment that may include a magnetic material. Where, the third segment may be placed between the first segment and the second segment. In one exemplary embodiment, the nanomotor may include a nanorod with a diameter of less than about 10 nm and a length of less than about 50 nm.

In one exemplary embodiment, the metal may include platinum (Pt), or palladium (Pd), or combinations thereof. In an exemplary embodiment, the magnetic material may include Nickel (Ni), or Cobalt, or combinations thereof.

In some exemplary implementations, forming the functionalized nanomotor by functionalizing the nanomotor with the aptamer may include binding the aptamer to the first segment of the nanomotor. In one exemplary embodiment, forming the functionalized nanomotor by functionalizing the nanomotor with the aptamer may include mixing a solution of the nanomotor with a solution of the aptamer for a period of time between about 10 hours and about 20 hours at a temperature of less than about 10 ° C.

In some exemplary implementations, the aptamer may include an anti-VEGF DNA aptamer, a Thrombin aptamer, a platelet-derived growth factor BB (PDGF-BB) aptamer, a Carcinoembryonic antigen (CEA), a Cytochrome c (CYC), a TNF-α aptamer, or combinations thereof. In one exemplary embodiment, the aptamer may include a modified aptamer with a functional thiolated (—SH) group, a functional amine group, or combinations thereof.

In some exemplary implementations, blocking gaps between functionalized parts of the functionalized nanomotor with the blocking agent may include immersing the functionalized nanomotor in a solution of the blocking agent. In one exemplary embodiment, the blocking agent may include 6-Mercapto-1-hexanol (MCH), or L-Cystine (L-cys), or Hexanethiol, or combinations thereof.

In some exemplary implementations, attaching the fluorescence probe to the blocked functionalized nanomotor may include binding the fluorescence probe to the aptamer by immersing the functionalized nanomotor in a solution of the fluorescence probe. In one exemplary embodiment, the fluorescence probe may include Methylene blue (MB).

In some exemplary implementations, forming the mixture by adding the suspicious biological solution to the reference solution may include guiding the biosensor by a magnetic field in the mixture. In one exemplary embodiment, the suspicious biological solution may include a Human serum sample.

In some exemplary implementations, measuring the first fluorescence intensity of the reference solution and measuring the second fluorescence intensity of the mixture may include measuring fluorescence intensity using a fluorescence spectroscopy technique. In one exemplary embodiment, the difference between the first fluorescence intensity and the second fluorescence intensity may include a greater value for the second fluorescence intensity in comparison with the first fluorescence intensity.

In another aspect of the present disclosure, a biosensor for tumor detection is disclosed. The biosensor may include a nanomotor, an aptamer, a blocking agent, and a fluorescence probe. The nanorod may have a diameter of less than about 50 nm and a length of less than about 100 nm and the nanorod may include a first segment including a golden (Au) segment. In one exemplary embodiment, the aptamer may be bound to the golden segment of the nanomotor, the blocking agent may be bound to unbound parts of the golden segment, and the fluorescence probe may be attached to the aptamer.

In some exemplary implementations, the nanorod may further include a second segment which may include platinum (Pt), or palladium (Pd), and a third segment, which may include Nickel (Ni), or cobalt. In one exemplary embodiment, the third segment may be placed between the first segment and the second segment.

In some exemplary implementations, the aptamer may include an anti-VEGF DNA aptamer, a Thrombin aptamer, a platelet-derived growth factor BB (PDGF-BB) aptamer, a Carcinoembryonic antigen (CEA), a Cytochrome c (CYC), a TNF-α aptamer, or combinations thereof. In one exemplary embodiment, the aptamer may include a modified aptamer with a functional thiolated (—SH) group, a functional amine group, or combinations thereof.

In some exemplary implementations, the blocking agent may include 6-Mercapto-1-hexanol (MCH), or L-Cystine (L-cys), or Hexanethiol, or combinations thereof. The the fluorescence probe may include Methylene blue (MB).

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A illustrates a method for tumor marker detection, consistent with one or more exemplary embodiments of the present disclosure.

FIG. 1B illustrates a method for preparing the biosensor, consistent with one or more exemplary embodiments of the present disclosure.

FIG. 2 illustrates a schematic view of an exemplary nanomotor, consistent with one or more exemplary embodiments of the present disclosure.

FIG. 3A illustrates an exemplary implementation of forming a functionalized nanomotor by functionalizing a nanomotor with an aptamer, consistent with one or more exemplary embodiments of the present disclosure.

FIG. 3B illustrates an exemplary implementation of blocking gaps between functionalized parts of the functionalized nanomotor with a blocking agent, consistent with one or more exemplary embodiments of the present disclosure.

FIG. 3C illustrates an exemplary implementation of attaching a fluorescence probe to the functionalized nanomotor, consistent with one or more exemplary embodiments of the present disclosure.

FIG. 4 illustrates an exemplary implementation of contacting an exemplary prepared biosensor with a suspicious biological solution that may contain a tumor marker, consistent with one or more exemplary embodiments of the present disclosure.

FIG. 5 illustrates a transmission electron microscopy (TEM) image of the nanomotors used as a substrate for the biosensor, consistent with one or more exemplary embodiments of the present disclosure.

FIG. 6A illustrates the fluorescence spectra of desorbed MB from biosensor in the absence (dashed line) and presence (solid line) of different concentrations of VEGF165 and the inset shows the visual color of released MB in presence of VEGF165 (30 nM), consistent with one or more exemplary embodiments of the present disclosure.

FIG. 6B illustrates the linear plot of relative fluorescence changes of the desorbed MB from biosensor at different from about 2.5 nM to about 30 nM, consistent with one or more exemplary embodiments of the present disclosure.

FIG. 7 illustrates relative fluorescence intensity of the desorbed MB from biosensor incubated in blank PBS buffer, glucose (5 mM), urea (5 mM), dopamine (5 mM), HIgG (20 nM), HSA (20 nM) and VEGF165 (20 nM) in PBS, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings. The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Catalytic nanomotors are nanoscale-manufactured devices which may be propelled by different mechanisms that basically convert chemical energy into autonomous motion. Exemplary biosensors, particularly, aptasensors may provide on-the-fly interaction with tumor markers and capturing thereof. Exemplary nanorod motors are effective and may be synthesized at low-costs allowing for their commercially and technically viable use in several applications, for example, tumor detection.

Herein, an exemplary biosensor including magnetically-guided Pt—Ni—Au nanomotors functioning as a substrate for immobilizing aptamers is disclosed for detection and diagnosis of tumor markers. Moreover, an exemplary method for tumor detection using the exemplary biosensor based on nanomotors is disclosed. The method may be capable of simple, fast, and accurate detecting tumor markers in a suspicious sample that may be assisted by a simple optical measurement. The method may include simultaneously monitoring fluorescence intensity of a solution of the biosensor before and after addition of the suspicious sample to the solution of the biosensor while magnetically guiding the biosensor within a mixture of the suspicious sample and the solution of the biosensor, so that providing a fast and simple method for tumor detection that may be responsive to a change of the fluorescence intensity of the suspicious sample.

In an aspect of the present disclosure, an exemplary method for tumor marker detection is disclosed. The method may be used for simple, fast, and label-free detection of a tumor in a suspicious sample, for example, Human serum sample. The method may not need complicated devices and may be designed based on the motion of nanomotors that may be used as a substrate for a biosensor, which may be applied in the present method.

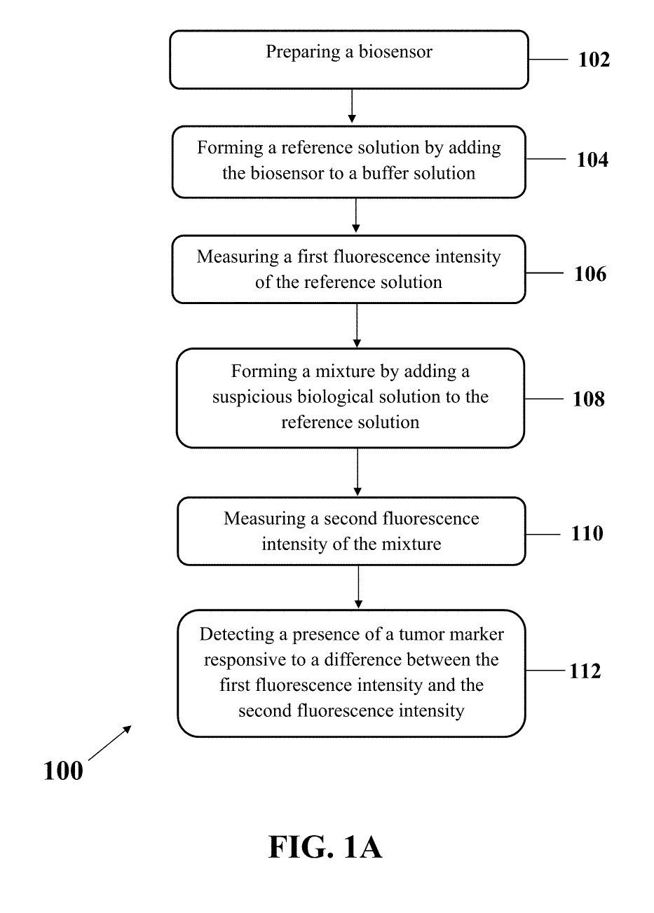

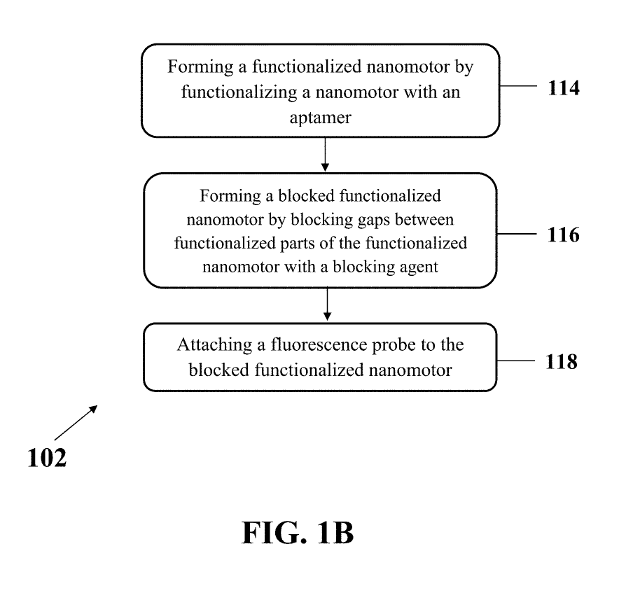

FIG. 1A shows a method 100 for tumor marker detection, consistent with exemplary embodiments of the present disclosure. Method 100 may include preparing a biosensor (step 102), forming a reference solution by adding the biosensor to a buffer solution (step 104), measuring a first fluorescence intensity of the reference solution (step 106), forming a mixture by adding a suspicious biological solution to the reference solution (step 108), measuring a second fluorescence intensity of the mixture (step 110), and detecting a presence of a tumor marker in the suspicious biological solution responsive to a difference between the first fluorescence intensity and the second fluorescence intensity (step 112)

FIG. 1B shows an exemplary implementation of preparing the biosensor (step 102) that may include forming a functionalized nanomotor by functionalizing a nanomotor with an aptamer (step 114), include forming a blocked functionalized nanomotor by blocking gaps between functionalized parts of the functionalized nanomotor with a blocking agent (step 116), and attaching a fluorescence probe to the functionalized nanomotor (step 118).

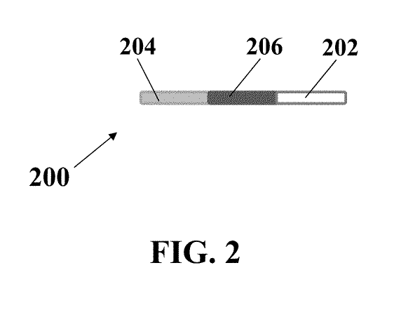

FIG. 2 shows a schematic view of an exemplary implementation of a nanomotor 200, consistent with one or more exemplary embodiments of the present disclosure. Nanomotor 200 may include a nanorod 200 with a diameter of less than about 50 nm and a length of less than about 100 nm. In an exemplary embodiment, nanomotor 200 may include the nanorod 200 with a diameter of less than about 10 nm and a length of less than about 50 nm. Nanorod 200 may include a first segment 202, a second segment 204, and a third segment 206; where the third segment 206 may be placed between the first segment 202 and the second segment 204. In an exemplary embodiment, the first segment 202 may include Gold (Au), the second segment 204 may include a metal, for example, platinum (Pt), or palladium (Pd), or combinations thereof, and the third segment 206 may include a magnetic material, for example, Nickel (Ni), or Cobalt, or combinations thereof.

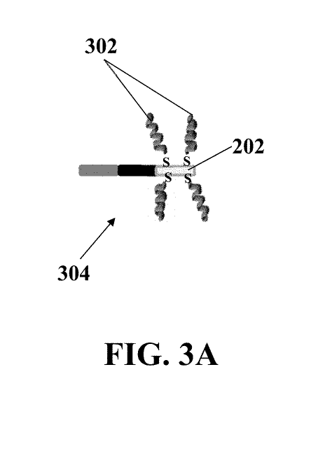

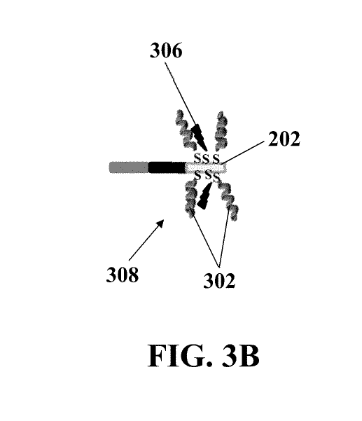

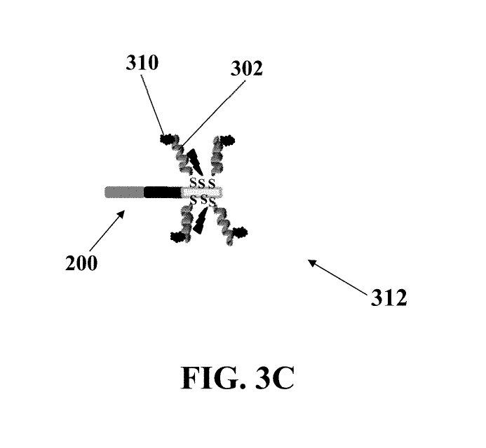

FIGS. 3A-3C show exemplary implementations of preparing an exemplary biosensor 312 (step 102), consistent with one or more exemplary embodiments of the present disclosure. FIG. 3A shows an exemplary implementation of forming a functionalized nanomotor 304 by functionalizing exemplary nanomotor 200 with an exemplary aptamer 302 (step 114), consistent with one or more exemplary embodiments of the present disclosure. In step 114, functionalized nanomotor 304 with aptamer 302 may be formed by functionalizing nanomotor 200 with aptamer 302. In an exemplary implementation, functionalized nanomotor 304 may be formed by binding aptamer 302 to the first segment 202 of nanomotor 200.

In an exemplary implementation, forming functionalized nanomotor 304 may include mixing a solution of nanomotor 200 with a solution of aptamer 302 for a period of time between about 10 hours and about 20 hours at a temperature of less than about 10° C., for example, in a refrigerator. Therefore, the aptamer 302 may be attached to the first segment 202 of nanomotor 200.

It should be noted that for selective tumor marker detection, an appropriate aptamer for each tumor marker may be used, for example, for detecting Vascular endothelial growth factor (VEGF165), thiolated ssDNA (anti-VEGF aptamer) may be used to attach to the first segment 202 of nanomotor 200. In an exemplary embodiment, the aptamer 302 may include a modified aptamer with a functional thiolated (—SH) group, or a functional amine group, or combinations thereof, so that the thiolated (—SH) group or the functional amine group of modified aptamer 302 may attach to the first segment 202 of nanomotor 200. In an exemplary embodiment, aptamer 302 may include anti-VEGF DNA aptamer, or Thrombin aptamer, or platelet-derived growth factor BB (PDGF-BB) aptamer, Carcinoembryonic antigen (CEA), Cytochrome c (CYC), TNF-α aptamer, or combinations thereof. Table 1 shows a list of examples of aptamer 302 and corresponding functional thiolated (—SH) groups.

TABLE 1
list of exemplary aptamers and corresponding functional thiolated (-SH)
groups

| aptamer | thiolated (-SH) group |
|---|---|
| anti-VEGF DNA aptamer | 5′ SH-TTTCCCGTCTTCCAGACAAGAGTGCAGGG-3′ |
| Thrombin aptamer | 5′ SH-GGT TGG TGT GGT TGG-3′ |
| platelet-derived growth factor | 5′SH-CAGGCTACGGCACGTAGAGCATCACCAT-GATCCTG-3 |
| BB (PDGF-BB) aptamer | |
| Carcinoembryonic antigen | 5′-SH-TTT TTT ATA CCA GCT TATTCA ATT-3′) |
| (CEA) | |
| cytochrome c (CYC) | 5′-SH- |
| | AGTGTGAAATATCTAAACTAAATGTGGAGGGTGGGACG |
| | GGAAGAAGTTTATTTTTCACACT-3 |
| TNF-α aptamer | 5′-SH- |
| | TTTTTTTTTTTTTTTTGGTGGATGGCGCAGTCGGCGACAA- |
| | 3′ |

FIG. 3B shows an exemplary implementation of blocking gaps between functionalized parts of functionalized nanomotor 304 with a blocking agent 306 (step 116), consistent with one or more exemplary embodiments of the present disclosure. In step 116, blocking agent 306 may be used to fill gaps between aptamers 302 to block all free parts of the first segment 202 of nanomotor 200; thereby, a blocked functionalized nanomotor 308 may be obtained. It should be noted that blocking gaps between functionalized parts of functionalized nanomotor 304 may be done because of that gold nanomaterials, for example, the first segment 202 of nanomotor 200, have high active area and therefore biological materials can interact with gold nanomaterials and change the analytical performance of functionalized nanomotor 304 for sensing a tumor marker. If other biomaterials except tumor markers interact with functionalized nanomotor 304, the sensitivity of functionalized nanomotor 304 and subsequently, the prepared biosensor to tumor markers would not be so good if this blocking agent is not present on the surface of nanomotor.

In an exemplary implementation, blocking gaps between functionalized parts of functionalized nanomotor 304 with the blocking agent 306 (step 116) may include immersing the functionalized nanomotor 304 in a solution of the blocking agent 306. In an exemplary embodiment, blocking agent 306 may include 6-Mercapto-1-hexanol (MCH), or L-Cystine (L-cys), or Hexanethiol, or combinations thereof.

FIG. 3C shows an exemplary implementation of attaching a fluorescence probe 310 to the blocked functionalized nanomotor 308 (step 118), consistent with one or more exemplary embodiments of the present disclosure. In step 118, fluorescence probe 310 may be attached to the blocked functionalized nanomotor 308 to form exemplary biosensor 312. In an exemplary embodiment, attaching fluorescence probe 310 to blocked functionalized nanomotor 308 may include binding the fluorescence probe 310 to aptamer 302; thereby, exemplary biosensor 312 may be obtained. In an exemplary embodiment, fluorescence probe 310 may bind to aptamer 302 by immersing blocked functionalized nanomotor 308 in a solution of fluorescence probe 310, so that exemplary biosensor 312 may be formed. In an exemplary embodiment, fluorescence probe 310 may include Methylene blue (MB), which may be able to bind to an aptamer.

In an exemplary implementation, biosensor 312 may be put in contact with a suspicious biological solution and a presence of a tumor marker in the suspicious biological solution may be detected through steps 104 to 112. The suspicious biological solution should be analyzed for a possible presence of a tumor marker and a change in fluorescent intensity may be monitored for tumor marker detection. In an exemplary embodiment, the suspicious biological solution may include a Human serum sample.

In step 104, a reference solution may be formed by adding biosensor 312 to a buffer solution. The formed reference solution may then be put in contact with a suspicious biological solution, which should be analyzed for a possible presence of a tumor marker. In an exemplary embodiment, biosensor 312 may be added to a phosphate buffer solution (PBS) to form the reference solution.

In step 106, a first fluorescence intensity of the reference solution may be measured. In an exemplary embodiment, the first fluorescence intensity of the reference solution may be measured using a fluorescence spectroscopy technique.

In step 108, a mixture may be formed by adding the suspicious biological solution to the reference solution that may be obtained from step 104. In an exemplary embodiment, biosensor 312 may be guided in the mixture by a magnetic field in order to move the biosensor 312 through the mixture and enhance a contact between biosensor 312 and the suspicious biological solution within the mixture.

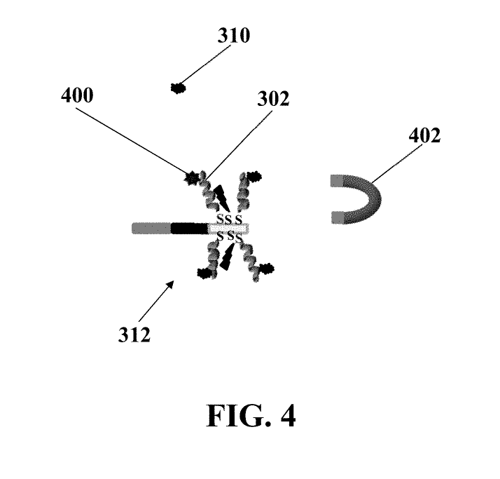

FIG. 4 shows an exemplary implementation of forming the mixture of the reference solution and the suspicious biological solution (step 104) that may provide contacting exemplary prepared biosensor 312 with the suspicious biological solution that may contain a tumor marker 400, consistent with one or more exemplary embodiments of the present disclosure. In an exemplary embodiment, forming the mixture of the reference solution containing biosensor 312 and the suspicious biological solution (step 104) may include guiding biosensor 312 by a magnetic field in the suspicious biological solution. In an exemplary embodiment, the magnetic field may be formed by exemplary magnet 402 that may induce an enhanced movement of biosensor 312 within the mixture so that an interaction between the aptamer 302 and the tumor marker 400 may be increased. Therefore, if a tumor marker 400 is present in the mixture, it may tend to attach to aptamer 302, resulting in releasing fluorescence probe 310 from the biosensor 312 due to substituting of tumor marker 400 for fluorescence probe 310 bound to aptamer 302. Hence, an increase in fluorescence intensity of the mixture in comparison with the reference solution may occur by releasing fluorescence probe 310 within the mixture.

In step 110, a second fluorescence intensity of the mixture may be measured in order to compare with the first fluorescence intensity of the reference solution; thereby, a presence of a tumor marker may be detected. In an exemplary implementation, second fluorescence intensity of the mixture may be measured using a fluorescence spectroscopy technique.

In step 112, a presence of a tumor marker may be detected responsive to a difference between the first fluorescence intensity and the second fluorescence intensity that may be identified by comparing the first fluorescence intensity of the reference solution measured in step 106 and the second fluorescence intensity of the mixture measured in step 110. In an exemplary embodiment, the presence of a tumor marker may be detected if an increase over a threshold amount in the fluorescent intensity is identified for the second fluorescent intensity in comparison with the first fluorescent intensity. The amount of difference between the first fluorescence intensity and the second fluorescence intensity and the threshold amount may depend on the concentration of the tumor marker in the suspicious biological solution and consequently, the concentration of the tumor marker in the mixture. In an exemplary embodiment, the difference between the first fluorescence intensity and the second fluorescence intensity may include a greater value for the second fluorescence intensity than the first fluorescence intensity.

In an exemplary embodiment of the present disclosure, a biosensor for tumor detection is disclosed, such as exemplary biosensor 312 (FIG. 3C) that may be prepared in step 102 of method 100 of the present disclosure. Referring to FIG. 3C, biosensor 312 may include a nanomotor, which may include the nanorod 200 including a first segment 202. An exemplary implementation of nanorod 200 is shown in FIG. 2. The biosensor may further include aptamer 302, blocking agent 306, and fluorescence probe 310. In an exemplary implementation, the aptamer 302 may be bound to the golden segment 202 of the nanomotor, the blocking agent 306 may be bound to unbound parts of the golden segment 202, and the fluorescence probe 310 is attached to the aptamer 302.

In an exemplary implementation, nanorod 200 may include a first segment 202 that may include a golden (Au) segment. Nanorod 200 may further include a second segment 204, which may include platinum (Pt), or palladium (Pd), and a third segment 206, that may include Nickel (Ni), or cobalt. The third segment 206 may be placed between the first segment 202 and the second segment 204. In an exemplary embodiment, nanorod 200 may have a size including a diameter of less than about 50 nm and a length of less than about 100 nm, for example, a diameter of less than about 10 nm and a length of less than about 50 nm.

In an exemplary implementation, the aptamer 302 may include anti-VEGF DNA aptamer, or Thrombin aptamer, or platelet-derived growth factor BB (PDGF-BB) aptamer, or Carcinoembryonic antigen (CEA), or Cytochrome c (CYC), or TNF-α aptamer, or combinations thereof. In an exemplary embodiment, aptamer 302 may include a modified aptamer with a functional thiolated (—SH) group, a functional amine group, or combinations thereof. In an exemplary embodiment, blocking agent 306 may include one of 6-Mercapto-1-hexanol (MCH), L-Cystine (L-cys), Hexanethiol, or combinations thereof. In an exemplary embodiment, fluorescence probe 310 may include Methylene blue (MB).

### EXAMPLE 1

### Biosensor for the Sensing of VEGF165 Tumor Marker

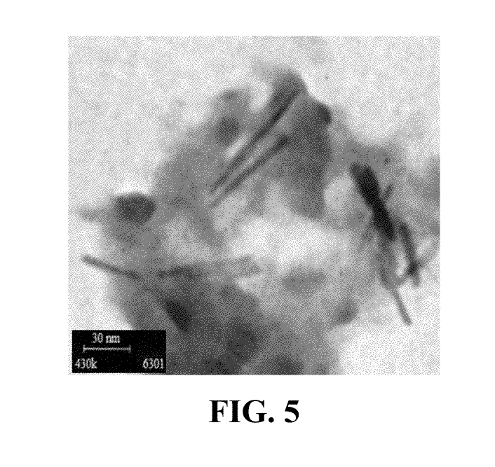

In this example, a biosensor for detecting VEGF165 tumor marker was prepared. For this purpose, nanomotors were synthesized and used as a substrate for the biosensor. FIG. 5 shows a transmission electron microscopy (TEM) image of the nanomotors used as the substrate for the biosensor, consistent with one or more exemplary embodiments of the present disclosure. The width of the nanomotors were estimated to be about 6 and the length of the nanomotors were estimated to be about 40 nm.

For preparing the biosensor, a solution of nanomotors were added to a 1 μM thiolated ssDNA (anti-VEGF aptamer modified at the 5′-terminus with an SH group, with a sequence of 5′-TTTCCCGTCTTCCAGACAAGAGTGCAGGG-3′) solution for about 18 hours and then soaked in the 1 mM 6-mercaptohexanol (MCH) solution for about 6 hours to fill any unoccupied gaps on the ion channel surface to prevent subsequent nonspecific adsorption. For fabrication of MB/aptamer/nanomotor, the functionalized nanomotor (0.5 mg mL⁻¹) with aptamer were immersed in phosphate buffer solution (PBS, 0.1 M) containing MB (25 μM) for about 15 minutes under stirring at room temperature. Then, the fabricated MB/aptamer/nanomotors were fixed by magnetic force on the wall of the vial and rinsed thoroughly with PBS several times to wash away the loosely adsorbed MB.

### EXAMPLE 2

### Detection of VEGF165 Tumor Marker by the Nanomotor-Based Biosensor

In this example, the fabricated MB/aptamer/nanomotors of EXAMPLE 1 were guided by a magnetic field into solutions (human serum samples) containing various concentration of VEGF165 to react with the fabricated MB/aptamer/nanomotor for about 40 minutes. MB can specifically bind with guanine bases in ss-DNA and the used aptamer herein as a type of ss-DNA riches of guanine bases. The fabricated MB/aptamer/nanomotors were stored at about 4° C. in the refrigerator when they were not in use. Then, the MB/aptamer/nanomotors biosensor was guided to solutions containing the various concentration of VEGF165 tumor marker. Upon exposing MB/aptamer/nanomotor with VEGF165, the adsorbed MB to aptamers released to the solution and let to increase the fluorescence signal of MB. The proposed nanomotor may be used for ‘on-the-fly’ interaction of biological targets such as VEGF165 by functionalizing the gold surface of the nanomotor with various bio-receptors.

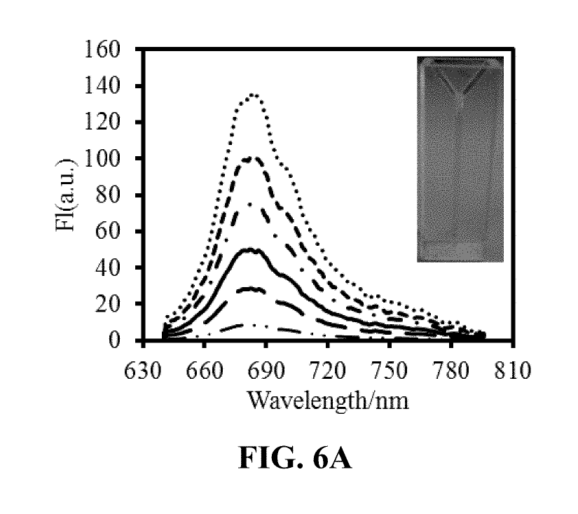

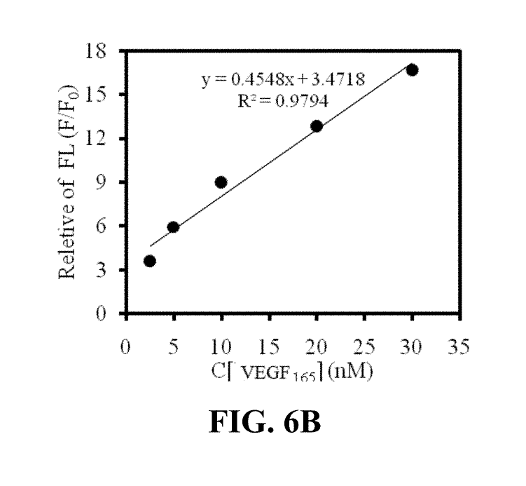

FIG. 6A shows the fluorescence spectra of desorbed MB from biosensor in the absence (dashed line) and presence (solid line) of different concentrations of VEGF165 and the inset shows the visual color of released MB in presence of VEGF165 (30 nM), consistent with one or more exemplary embodiments of the present disclosure.

FIG. 6B shows the linear plot of relative fluorescence changes of the desorbed MB from biosensor at different from about 2.5 nM to about 30 nM, consistent with one or more exemplary embodiments of the present disclosure. In FIG. 6B, relative fluorescence changes are calculated by F/F0, where F0 (7.8) and F are the fluorescence intensity without and with VEGF165, respectively, and excitation wavelength was 625 nm. FIG. 6B shows a calibration curve that indicates the dependence of fluorescence signal to the concentration of VEGF165 within the solution. A linear relation between x (concentration of VEGF165 within the solution) and y (fluorescence signal due to a release of MB from the associated aptamer) shows a linear relation with a formula of:

y=0.4548x+3.4718

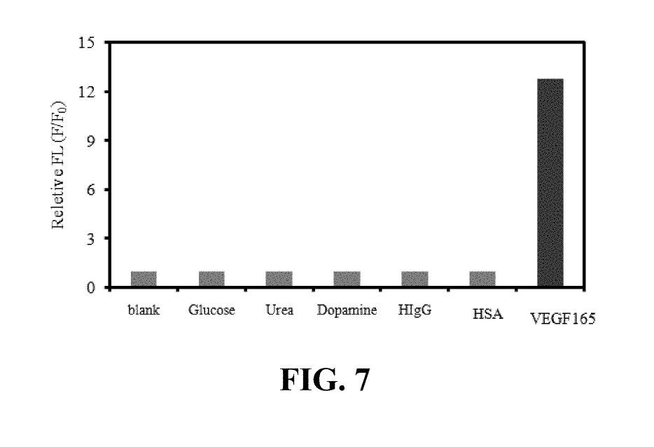

In addition, to evaluate the selectivity of the proposed biosensor, some of bio-molecules such as human serum albumin (HSA), bovine serum albumin (BSA), glucose (G), urea (U), dopamine (D) and human immunoglobulin G (HIgG) were used as the potential interferences to evaluate the specificity and the results were shown in FIG. 7. FIG. 7 shows relative fluorescence intensity of the desorbed MB from biosensor incubated in blank PBS buffer, glucose (5 mM), urea (5 mM), dopamine (5 mM), HIgG (20 nM), HSA (20 nM) and VEGF165 (20 nM) in PBS, consistent with one or more exemplary embodiments of the present disclosure. As indicated, the proposed biosensor possesses a suitable selectivity towards VEGF165.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1- A method for tumor marker detection, the method comprising:
preparing a biosensor, comprising
forming a functionalized nanomotor by functionalizing a nanomotor with an aptamer;
forming a blocked functionalized nanomotor by blocking gaps between functionalized parts of the functionalized nanomotor with a blocking agent; and
attaching a fluorescence probe to the blocked functionalized nanomotor;
forming a reference solution by adding the biosensor to a buffer solution;
measuring a first fluorescence intensity of the reference solution;
forming a mixture by adding a suspicious biological solution to the reference solution;
measuring a second fluorescence intensity of the mixture; and
detecting a presence of a tumor marker responsive to a difference between the first fluorescence intensity and the second fluorescence intensity.

2- The method of claim 1, wherein the nanomotor comprises a nanorod with a diameter of less than 50 nm and a length of less than 100 nm, the nanorod comprising:
a first segment, the first segment comprises Gold (Au);
a second segment, the second segment comprises a metal; and
a third segment, the third segment comprises a magnetic material,
wherein the third segment is placed between the first segment and the second segment.

3- The method of claim 2, wherein the nanomotor comprises a nanorod with a diameter of less than 10 nm and a length of less than 50 nm.

4- The method of claim 2, wherein the metal comprises one of platinum (Pt), palladium (Pd), or combinations thereof.

5- The method of claim 2, wherein the magnetic material comprises one of Nickel (Ni), Cobalt, or combinations thereof.

6- The method of claim 2, wherein forming the functionalized nanomotor by functionalizing the nanomotor with the aptamer comprises binding the aptamer to the first segment of the nanomotor.

7- The method of claim 1, wherein forming the functionalized nanomotor by functionalizing the nanomotor with the aptamer comprises mixing a solution of the nanomotor with a solution of the aptamer for a period of time between 10 hours and 20 hours at a temperature of less than 10° C.

8- The method of claim 1, wherein the aptamer comprises a modified aptamer with one of a functional thiolated (—SH) group, a functional amine group, or combinations thereof.

9- The method of claim 1, wherein blocking gaps between functionalized parts of the functionalized nanomotor with the blocking agent comprises immersing the functionalized nanomotor in a solution of the blocking agent, and
wherein the blocking agent comprises 6-Mercapto-1-hexanol (MCH), L-Cystine (L-cys), Hexanethiol, or combinations thereof.

10- The method of claim 1, wherein attaching the fluorescence probe to the blocked functionalized nanomotor comprises binding the fluorescence probe to the aptamer by immersing the functionalized nanomotor in a solution of the fluorescence probe.

11- The method of claim 1, wherein the fluorescence probe comprises Methylene blue (MB).

12- The method of claim 1, wherein forming the mixture by adding the suspicious biological solution to the reference solution comprises guiding the biosensor by a magnetic field in the mixture.

13- The method of claim 1, wherein the suspicious biological solution comprises a Human serum sample.

14- The method of claim 1, wherein measuring the first fluorescence intensity of the reference solution and measuring the second fluorescence intensity of the mixture comprises measuring fluorescence intensity using fluorescence spectroscopy technique.

15- The method of claim 1, wherein the difference between the first fluorescence intensity and the second fluorescence intensity comprises a greater value for the second fluorescence intensity than the first fluorescence intensity.

16- A biosensor for tumor detection, the biosensor comprising:
a nanomotor comprising a nanorod with a diameter of less than 50 nm and a length of less than 100 nm, the nanorod comprising a first segment comprising a golden (Au) segment;
an aptamer, the aptamer bound to the golden segment of the nanomotor;
a blocking agent, the blocking agent bound to unbound parts of the golden segment; and
a fluorescence probe attached to the aptamer.

17- The biosensor of claim 16, wherein the nanorod further comprises:
a second segment comprising one of platinum (Pt) or palladium (Pd); and
a third segment comprising one of Nickel (Ni) or cobalt; wherein the third segment is placed between the first segment and the second segment.

18- The biosensor of claim 16, wherein the aptamer comprises the aptamer comprises one of anti-VEGF DNA aptamer, Thrombin aptamer, platelet-derived growth factor BB (PDGF-BB) aptamer, Carcinoembryonic antigen (CEA), Cytochrome c (CYC), TNF-α aptamer, or combinations thereof.

19- The biosensor of claim 16, wherein the aptamer comprises a modified aptamer with one of a functional thiolated (—SH) group, a functional amine group, or combinations thereof, and
wherein the blocking agent comprises one of 6-Mercapto-1-hexanol (MCH), L-Cystine (L-cys), Hexanethiol, or combinations thereof.

20- The biosensor of claim 16, wherein the fluorescence probe comprises Methylene blue (MB).
